(19) United States
(12) Patent Application Publication
(10) Pub. No.: US 2007/0191465 A1
(43) Pub. Date: Aug. 16, 2007

(54) Channel blocking compounds

(75) Inventors: Julie Eleanor Dalziel, Palmerston North (NZ); James Dunlop, Palmerston North (NZ); Sarah Christine Munday-Finch, Hamilton (NZ)

Correspondence Address: KNOBBE MARTENS OLSON & BEAR LLP, 2040 MAIN STREET, FOURTEENTH FLOOR, IRVINE, CA, 92614, US

(73) Assignee: Agresearch Limited, Hamilton (NZ)

(21) Appl. No.: 10/564,872

(22) PCT Filed: Aug. 13, 2004
(86) PCT No.: PCT/NZ04/00184

§ 371 Date: Feb. 14, 2007

(30) Foreign Application Priority Data
Aug. 15, 2003 (NZ) 527607

(51) Int. Cl. A61K 31/407

(52) U.S. Cl. 514/422; 514/410

(57) ABSTRACT

The invention relates to compositions and associated methods and uses that contain lolitrem compounds containing the moiety shown in structure (I) or derivatives thereof for ion channel antagonist applications, particularly BK channel antagonist applications. Preferred compounds include lolitrem B, 31-epilolitrem B, lolitriol and lolitrem E. For lolitrem B, a particularly strong and long acting blocking effect is identified.

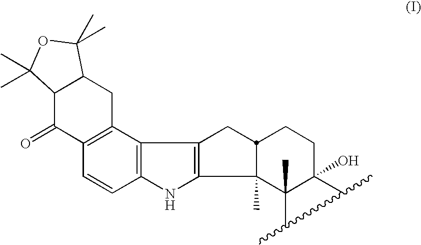

### TECHNICAL FIELD

The present invention relates to ion channel blocking compounds in particular, compositions containing alkaloid compounds termed lolitrems for use as ion channel antagonists and to methods and uses of these compositions. More specifically, the lolitrem compounds are derived from the species Neotyphodium (formally Acremonium) lolii and are used as potassium channel antagonists.

### BACKGROUND ART

Ion channels are defined as transmembrane pores that present a central aqueous pore that can be opened by conformational change to allow ions to cross a lipid bilayer down their electrochemical gradients. Some degree of ion specificity is usually observed and typically a million ions per second flow through the channel. Channels may open spontaneously, like the potassium leak channel, or they may be voltage-gated, like the voltage-gated sodium channel or ligand-gated, like the acetylcholine receptor.

Ion channels generally are the subject of much research to understand the roles they have in normal physiological systems and in disease states.

Potassium ion channels are selective for potassium ions. There are diverse types of potassium ion channels with different functions, for example: voltage-gated potassium channels, delayed rectifier channels, M channels, A channels, inward rectifier channels, and calcium-activated potassium channels.

Large or high conductance calcium-activated potassium channels are also termed as BK channels, K_{Ca}, maxi-K. Slowpoke (Slo) is the name of the gene that encodes the pore-forming α subunit of the channel, e.g. hSlo—human gene, mSlo—mouse, dSlo drosophila—fruit fly. Accessory β subunits (β₁-β₄) associate with the a subunit to generate BK channel diversity. BK channels are gated by Ca⁺⁺ and membrane potential with a unit conductance of 100 to 300 picoSiemens (pS)

Calcium-activated potassium channels also include intermediate conductance (IK) and small conductance (SK) channels. IK potassium channels are more sensitive to Ca⁺⁺ than BK channels and are gated only by internal Ca⁺⁺ ions, having a unit conductance of 25 to 100 pS. SK channels are also highly sensitive to Ca⁺⁺ and have minimal voltage sensitivity, and a unit conductance of 2 to 25 pS.

For the purposes of this specification, the term ‘BK channel’ or ‘potassium channel’ or similar variations will be referred to although, this should not be seen as limiting.

BK channels are expressed in many tissues, including muscle and brain and regulate important physiological functions (for review see Gribkoff et al., 2001a). They have a role in regulation of blood pressure and are implicated in hypertension (Brenner et el., 2000a, Amberg and Santana, 2003, Amberg et al., 2003). They are activated in response to depolarising voltages and to increased intracellular calcium. Their activation results in efflux of potassium ions causing hyperpolarisation which dampens cellular excitability. BK channels are expressed in most tissues and control a large variety of physiological processes including smooth muscle tone, neurosecretion and hearing.

In blood vessels, BK channels oppose vasoconstriction, allowing vasorelaxation and thereby regulate arterial tone (i.e. blood pressure) (Nelson et al., 1995).

In the brain, they modulate action potential waveform, repetitive firing and neurotransmitter release (Shao et al., 1999, Golding et al., 1999, Hu et al., 2001). They are also expressed in the cochlea of the ear where they have a specialised role in frequency tuning of hair cells, acting in concert with other ion channels (Gribkoff, et al 2001a; Orio et al 2002).

BK channels are also expressed in other tissues where their role is not known, e.g. ovary, testis and kidney (Brenner et al., 2000b).

Compounds that block (inhibit) a biologic activity or process in this transfer of ions across a cell membrane are called ‘blockers’. They may also be termed ‘antagonist compounds’ as the compounds reduce or prevent ion transfer. For the purposes of this specification the term ‘antagonist’ will be used. This should not, however be interpreted as limiting.

The function of BK channels is modulated by a variety of compounds (for review see Kaczorowski and Garcia, 1999, Kaczorowski et al., 1996).

Known marketed drugs that block potassium channels include Glyburide™, Glipizide™ and Tolbutamide™. Other naturally occurring toxins that are known to block potassium channels include Apamin, Iberiotoxin, Charybdotoxin, Noxiustoxin and Kaliotoxin. U.S. Pat. No. 5,541,208 describes uses of these blockers and the use of paxilline, a further blocking compound, and is incorporated herein by reference.

Lolitrem compounds belong to the broader group of alkaloid compounds incorporating indole diterpenes.

Assay techniques for identifying lolitrem compounds are known, for example see NZ 236879.

Lolitrem compounds are present in perennial ryegrass (Lolium perenne) infected with the endophytic fungus Neotyphodium (formally Acremonium) lolii (Lane et al., 2000). Lolitrem compounds have been extracted from endophyte-infected ryegrass seed (Gallagher et al., 1981, Miles et al., 1994, Munday-Finch et al., 1995, Munday-Finch et al., 1996, Munday-Finch et al., 1997, Munday-Finch et al., 1998).

Endophytes are symbiotic fungi and are prevalent in at least New Zealand pastures. The fungal metabolites from these endophytes are thought to serve as chemical defence systems for the fungi that produce them. They may also be of use in protecting the food source from consumption by other organisms (U.S. Pat. No. 4,973,601).

The lolitrems are neurotoxic indole-diterpenes and are the principal causative agents of ryegrass staggers. This is a condition in which animals grazing on endophyte infected ryegrass-dominant pastures develop ataxia, tremors, and hypersensitivity to external stimuli. The lolitrem neurotoxin (staggers) reaction is long acting but is however completely reversible (Smith et al 1997, McLeay et al 1999). The time course of tremors induced by lolitrem B is dramatically different from that of other indole diterpenes, for example paxilline and analogues. When injected into mice, paxilline analogues induce tremors of rapid onset and short duration while tremors induced by lolitrem derivatives take hours to reach maximum intensity and last for days (Munday-Finch, 1997). Tremors induced by lolitrem are also longer in duration than those induced by the indole diterpene, aflatrem (Gallagher and Hawkes, 1996).

Whilst at least some lolitrems and other indole diterpenes, for example paxilline, are known to cause tremorgenicity (tremorgenic mycotoxins) there is no proven link between tremorgenicity and BK channel blocking.

Some linkage is inferred between tremorgenicity and neurotransmitter release (Mantle 1983, Gallagher et al 1986, Smith et al 1997, McLeay et al 1999, Wang et al 2003).

Some alkaloid compounds and more specifically indole diterpenes, block BK channels (e.g. paxilline, U.S. Pat. No. 5,541,208) and some do not (McMillan et al., 2003). The alkaloids that inhibit BK channels include both tremorgens and non-tremorgens. Structural moieties that are important for BK channel antagonism have been determined for some paxilline derivatives (Knaus et al 1994). However, for other types of indole diterpenes (e.g. lolitrems) whether a given compound will inhibit the BK channel cannot yet be predicted from structure alone but must be determined empirically.

Within a given structural class of indole diterpene, tremorgenicity cannot be predicted by structure. For example, while paxilline and lolitrem B are tremorgenic, lolilline, which is intermediate in structure between the two, is non-tremorgenic.

The structural features required for tremorgenicity are also different for each group of structurally related indole diterpenoid compounds. An acetal-linked isoprene unit, the presence of A/B rings and the stereochemistry at the A/B ring junction have been identified as important structural features for the tremorgenicity of lolitrem derivatives. Different structural features are required for tremorgenicity for other indole diterpene compounds.

As channel blockers have a variety of pharmaceutical uses and have been found to be beneficial for treatment of some diseases (for example Parkinson's disease, U.S. Pat. No. 5,541,208), such blocking compounds are of interest, particularly in the development of new therapies. A BK channel modulator that opens channels has been investigated as a neuroprotective drug in treating ischemic stroke (Gribkoff et al., 2001b).

It is an object of the present invention to provide an alternative ion channel blocking compound or at least to provide the public with a useful choice.

All references, including any patents or patent applications cited in this specification are hereby incorporated by reference. No admission is made that any reference constitutes prior art. The discussion of the references states what their authors assert, and the applicant reserves the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

Further aspects and advantages of the present invention will become apparent from the ensuing description which is given by way of example only.

### DISCLOSURE OF THE INVENTION

It has been found by the inventors that lolitrem compounds are antagonists of potassium (BK) ion channels.

For the purposes of this specification the term ‘ion channel’ refers to transmembrane pores that present a central aqueous pore that can be opened by conformational change to allow ions to cross a lipid bilayer down their electrochemical gradients.

For the purposes of this specification the term ‘antagonist’ refers to compounds that reduce or prevent ion transfer across a cell membrane.

According to one aspect of the present invention there is provided a composition that contains a pharmacologically effective amount of at least one BK channel antagonist compound containing the moiety shown in structure (I): 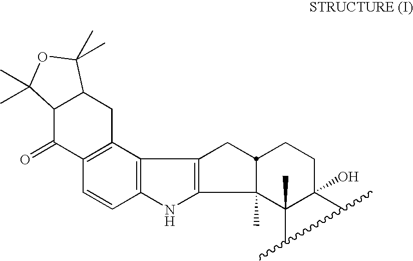
or derivatives thereof.

Preferably, derivatives of structure (I) are selected from the group consisting of: salts, analogues, isomers, and combinations thereof.

Preferably, the antagonist compound is selected from the group consisting of: lolitrem B, lolitrem A, lolitrem F, 31-epilolitrem F, 31-epilolitrem B, lolitrem E, lolitrem E acetate, lolitrem L, lolitrem G, lolitrem C, lolitrem M, lolitriol, lolitriol acetate, lolitrem N, lolitrem J, lolitrem H, lolitrem K, lolicine A and B, 30-desoxy lolitrem B-30α-ol, 30-desoxy-31-epilolitrem B-30α-ol, 30-desoxylolitrem B-30-ene lolilline and combinations thereof.

Preferably, the antagonist compound is structure (II): 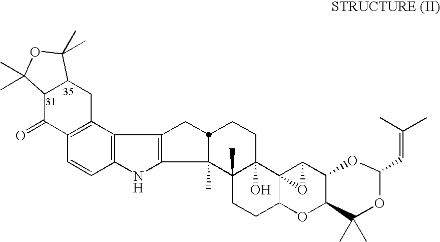
which includes compounds selected from the group consisting of: lolitrem B=31α, 35β stereochemistry; 31-epilolitrem B=31β, 35β stereochemistry; lolitrem F=31α, 35α; 31-epilolitrem F=31β, 35α.

Preferably, the antagonist compound is structure (III): 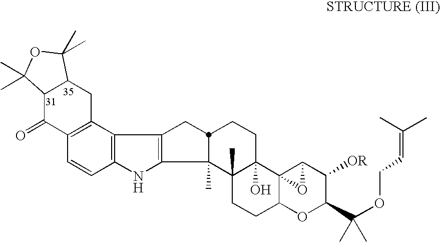
which includes compounds selected from the group consisting of: lolitrem E=31α, 35β stereochemistry where R=H or acetate; lolitrem L=31α, 35α stereochemistry where R=H or acetate.

Preferably, the antagonist compound is structure (IV): 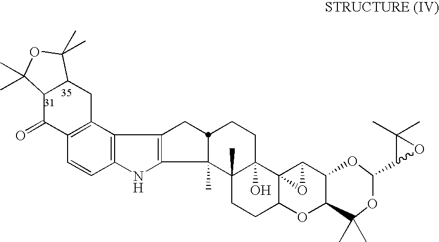
which includes compounds selected from the group consisting of: lolitrem A=31α, 35β stereochemistry; lolitrem G=31α, 35α stereochemistry.

Preferably, the antagonist compound is structure (V): 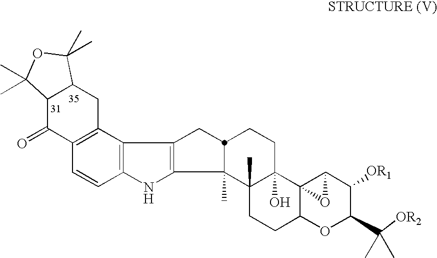
which includes compounds selected from the group consisting of: lolitriol;=31α, 35β stereochemistry where R₁=H or acetate and R₂=H; lolitrem N=31α, 35α stereochemistry where R₁=H or acetate and R₂=H; Lolitrem J=31α, 35β stereochemistry where R₁=H or acetate and R₂=acetate.

Preferably, the antagonist compound is structure (VI): 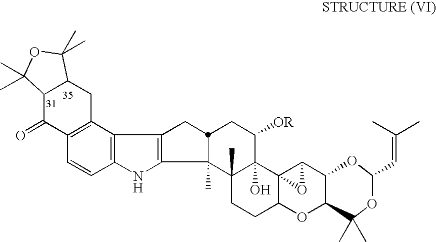
which includes lolitrem H=31α, 35β stereochemistry where R=H or acetate.

Preferably, the antagonist compound is structure (VII): 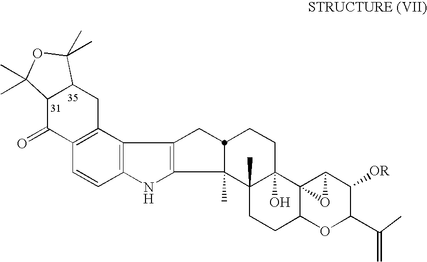
which includes lolitrem K=31α, 35β stereochemistry, where R=H or acetate.

Preferably, the antagonist compound is structure (VIII): 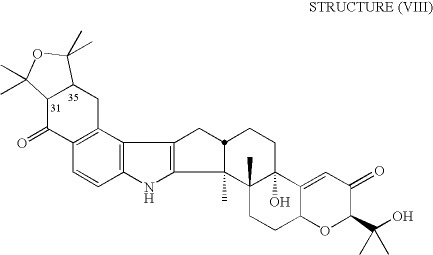
which includes lolilline=31α, 35β stereochemistry.

Preferably, the antagonist compound is structure (IX): 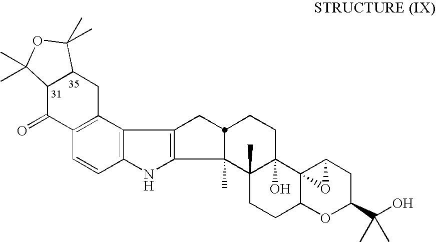
which includes lolitrem M=31α, 35β stereochemistry.

Preferably, the antagonist compound is structure (X): 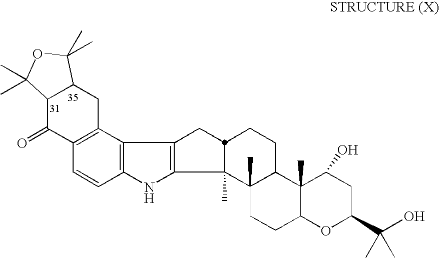
which includes lolicine A=31α, 35β stereochemistry.

Preferably, the antagonist compound is structure (XI): 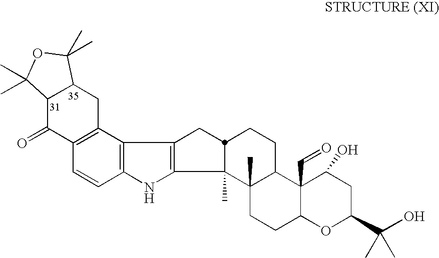
which includes lolicine B=31α, 35β stereochemistry.

Preferably, the antagonist compound is structure (XII): 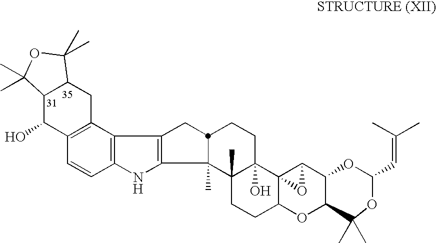
which includes compounds selected from the group consisting of: 30-desoxylolitrem B-30α-ol=31α, 35β stereochemistry; 30-desoxy-31-epilolitrem B-30α-ol=31 β, 35 β stereochemistry.

Preferably, the antagonist compound is structure (XIII): 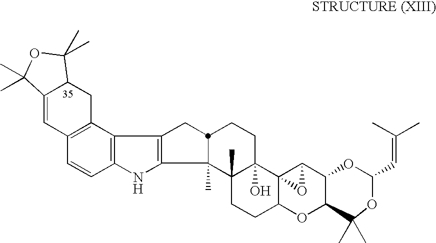
which includes 30-desoxylolitrem B-30-ene=35β stereochemistry.

Preferably, the composition further includes pharmaceutically and physiologically acceptable carriers. Preferably, the pharmaceutically and physiologically acceptable carriers include components selected from the group including; fillers; excipients; modifiers; humectants; stabilisers; emulsifiers; diluents; and other formulation components such as a use of a lipid vehicle.

Preferably, the composition, substantially as described above, is administered in a form selected from the group including: an injection; a tablet; a capsule; a suppository; an injection; a suspension; a drink or tonic; a syrup; a powder; an ingredient in solid or liquid foods; a nasal spray; a sublingual wafer; a transdermal patch; a transdermal injection; and combinations thereof. However, other methods of administration may also be employed without limiting the scope of the present invention.

Preferably, the BK channel antagonist compound or compounds are extracted from endophyte-infected plants and seeds; fungal cultures; chemical synthesis; heterologous expression systems including but not limited to bacteria, yeast, fungi, plants and animal cells; and combinations thereof.

In preferred embodiments, the source is perennial ryegrass seed from Lolium perenne. Further reference to lolitrem sources may be found in the applicants co-pending application NZ 530331.

Preferably, the ion channel is a potassium channel. More preferably, the potassium channel is a large conductance calcium activated potassium (BK) channel. Embodiments including intermediate conductance (IK) and small conductance (SK) may also be incorporated herein.

Preferably, the antagonist compound or compounds have activity against the alpha (α) subunit. More preferably, the antagonist compound or compounds have activity against both alpha (α) subunit and alpha plus beta (β) accessory subunit (β₁ to β₄) channels.

Preferably, for lolitrem B, the degree of antagonist inhibition is approximately 97% for a composition containing approximately 20 nM lolitrem B. The half maximal degree of antagonist inhibition (IC₅₀) is found for a composition containing approximately 3.7±0.4 nM of lolitrem B.

Preferably, for lolitriol, the degree of antagonist inhibition is approximately 100% for a composition containing approximately 1000 nM lolitriol. The half maximal degree of antagonist inhibition (IC₅₀) is found for a composition containing approximately 195 nM of lolitriol to inhibit a and β₁, activity and 536±16 nM of lolitriol to inhibit α and β₄ activity.

Preferably, for 31-epilolitrem B, the degree of antagonist inhibition is approximately 100% for a composition containing approximately 200 nM 31-epilolitrem B. The half maximal degree of antagonist inhibition (IC₅₀) is found for a composition containing approximately 58±6 nM of 31-epilolitrem B to inhibit α and β₁ activity and 49 nM of 31-epilolitrem B to inhibit α and β₄ activity.

Preferably, for lolitrem E, the degree of antagonist inhibition is approximately 100% for a composition containing approximately 100 nM lolitrem E.

It is the inventors understanding that the above levels of antagonist behaviour found in in vitro experiments indicates that lolitrem compounds have a high apparent affinity for at least hSlo channels. That is, the lolitrem compounds reduce and inhibit potassium currents through hSlo channels as well as other Slo channels including but not limited to mSlo and dSlo.

The antagonist effect of the composition is not able to be reversed at larger concentrations by wash out for at least lolitrem B. It is inventors experience that this effect is for concentrations of 10 nM or greater of lolitrem B compound.

According to a further aspect of the present invention there is provided a method of preventing repolarisation or hyperpolarisation of a cell, wherein the cell contains a BK channel, including the administration to the cell of a pharmacologically effective amount of composition containing a BK channel antagonist substantially as described above.

According to a further aspect of the present invention there is provided the use of a composition substantially as described above for preventing repolarisation or hyperpolarisation of a cell, wherein the cell contains a BK channel.

It should be appreciated from the above description that there are provided compositions, methods and uses incorporating lolitrem compounds to antagonise or block ion channel activity, particularly the BK channel.

It should further be appreciated that the blocking effect found for lolitrem compounds may be used in a variety of pharmaceutical applications such as for regulation of physiological functions including blood pressure, hypertension, muscle tone, brain functions such as neurotransmitter release and hearing application. In addition, lolitrem compounds, and particularly lolitrem B, have been found to have a blocking effect that is strong and long acting. Further applications envisaged for this blocking effect includes use in drug development and diagnostics i.e. a known blocking effect is generated from lolitrem compounds which may be used to find new drugs or to test if various physiological effects are altered from blocked channels. The above applications should not be seen as limiting as it should be appreciated by those skilled in the art that other applications may also be possible.

### BRIEF DESCRIPTION OF THE DRAWINGS

Further aspects of the present invention will become apparent from the following description which is given by way of example only and with reference to the accompanying drawings in which:

FIG. 1 shows two current recordings (A and B) for different concentrations of lolitrem B compared to control recordings of (A) ramp potential and (B) depolarising voltage pulses to +150 mV to determine the degree of antagonist inhibition;

FIG. 2 shows a graph of inhibition produced by different concentrations of lolitrem B. I/Imax is the current response as a fraction of the control response;

FIG. 3 shows two graphs (A and B) illustrating the effect of lolitrems on BK channel potassium currents for 31-epilolitrem B;

FIG. 4 shows two graphs (A and B) illustrating the effect of lolitrems on BK channel potassium currents for lolitriol and channels containing different beta subunits; and

FIG. 5 shows a graph illustrating dose-response curves for macroscopic BK channel potassium currents inhibited by lolitrems. Current amplitude (I′) 5 minutes after addition of increasing lolitriol concentrations, shown as a fraction of the control response. Lolitriol was applied to α+β₁ channels (solid circles) and to α+β₄ channels (open circles). 31-epilolitrem B was applied to α+β₁ channels (solid triangles) and to α+β₄ channels (open triangles). Shaded triangles are data points from a single cell to which the curve was fitted. Lolitrem B was applied to hSlo channels (solid squares) a previous study and is shown for comparison. The current response was averaged over the last half of a voltage pulse to +150 mV for 50 ms, with 10 μM internal free calcium. The vertical bars show ±1 S.E.M. in 3 or more cells. The curve is a fit of a Hill-type equation to the data.

### BEST MODES FOR CARRYING OUT THE INVENTION

The results found from experiments carried out by the inventors are now described.

Experiment 1

In this experiment, hSlo α subunit large conductance calcium-activated potassium (BK) channels with an N-terminal c-myc tag in the mammalian vector pcDNA (Meera et al, 1997) were transiently expressed in human embryonic kidney cells (cell type HEK293).

Cell Culture Preparation

Human embryonic kidney cells were grown in a mix of DMEM (Dulbecco's Modified Eagle Medium, GibcoBRL Cat#12100-038) and 2.5 mM HEPES (N-[2-Hydroxyethyl]piperazine-N′-[2-ethanesulfonic acid]), supplemented with minimal essential amino acids and 10% fetal bovine serum.

Cells were subsequently plated into 24-well plates, grown to 95% confluency and transfected 24 hours later with 10 μg of hSlo and 2 μg CD4 (pcDNA) and 2 82 l Lipofectamine 2000™.

Cells were plated onto cover slips 24 hours later. CD4 antibody-labelled beads were used to identify transfected cells. Macroscopic currents were recorded from excised inside-out patches at 3 days post-transfection.

Lolitrem B Preparation

Lolitrem B was extracted from perennial ryegrass seed infected with Neotyphodium lolii.

A stock of 100 μM Lolitrem B was made up in dimethyl sulfoxide (DMSO). This was diluted to the appropriate concentration in electrophysiological solutions. The final DMSO concentration was 0.1% for 100 nM lolitrem B and did not exceed 0.02 % for lower concentrations.

Electrophysiology

Solutions

The bath solution that was applied to the internal side of the cell membrane of the inside-out membrane patch was (mM): 140 KMeSO₃, 2 KCl, 20 HEPES (N-[2-Hydroxyethyl]piperazine-N′-[2-ethanesulfonic acid]), 5 HEDTA (N-([2-hydroxyethyl)ethylene-diaminetriacetic acid) and 3.65 CaCl₂ to give 10 μM free calcium with a pH of 7.2.

The pipette solution applied to the extemal side of the cell membrane of the inside-out membrane patch was (mM): 140 KMeSO₃, 2 KCl, 20 HEPES, and 2 MgCl₂, with a pH of 7.2.

Macroscopic currents were recorded in an inside-out patch-clamp configuration using an amplifier, interface and data collection software. Data were filtered at 5 kHz and sampled at 20 μs intervals. Fast capacitance compensation was used to cancel the fast transient. Leak subtraction was used although the background potassium current was small.

Results

Inhibition of BK channels by lolitrem B

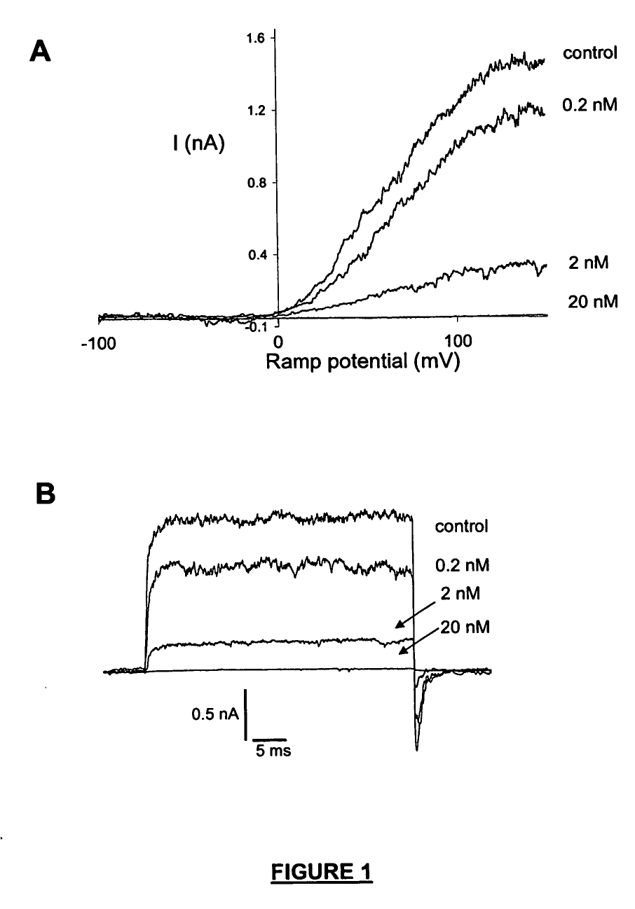

The effect of lolitrem B on potassium currents in excised inside-out patches from cells expressing hSlo BK channels is shown in FIG. 1. The Figure shows two current recordings (A and B) for different concentrations of lolitrem B compared to control recordings of (A) ramp potential and (B) depolarising voltage pulses to +150 mV to determine the degree of antagonist inhibition.

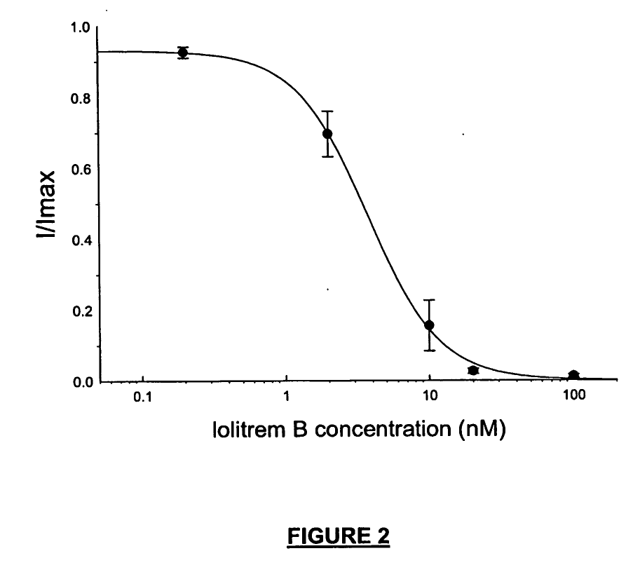

In FIG. 2, solid circles show the mean normalised current (fraction blocked) in three or more cells and the vertical bars are mean ±1 S.E.M. The curve is a fit of the Hill equation to the data.

FIG. 2 shows that the application of 20 nM lolitrem B to the perfusion bath, resulted in complete inhibition of the BK channel current. The level of antagonist inhibition was less at lower lolitrem B concentrations.

Channels were activated by voltage pulses to +150 mV every minute in the presence of 10 μM free calcium. Control current responses were recorded over 5 minutes. Only patches that remained stable over this time were used in experiments.

The current block produced by 10 nM or greater lolitrem B could not be reversed, even after wash-out with control solution for 30 minutes at a flow rate of 4 ml/min in three experiments.

At 2 nM lolitrem B partial inhibition was observed.

Dose-response experiments were carried out to determine the concentration range over which lolitrem B was effective.

Recovery from inhibition could not be used to validate reductions in current at different lolitrem B concentrations as being due to the presence of drug, nor could different concentrations be applied in random order. However, by applying cumulative doses of lolitrem B to the same membrane patch, it was found that increases in the degree of current block with increased lolitrem B concentration were consistent between cells.

Each concentration of lolitrem B was applied for 5 minutes or until the current response had stabilised and fractional block calculated as the decrease in current as a fraction of the control. The current amplitude was the mean current over the last half of the voltage pulse to +150 mV. The data was analysed and fitted using the Hill equation which gave an estimate of the concentration of half maximal inhibition (IC₅₀) of 3.7±0.4 nM and a Hill coefficient of 1.7, from experiments using 11 cells.

The concentration range of inhibition observed for lolitrem B is similar to that reported for other indole diterpenes including: paxilline, aflatrem, penitrem A, paspalinine, paspalitrems A and C, verruculogen and paspalicine applied to BK channels (Knaus et al., 1994, Sanchez and McManus, 1996, Gribkoff et al., 1996).

Difficulty in reversing channel block is also noted for these compounds, although paxilline block by low concentrations could be partially reversed by washout (Knaus et al., 1994).

Thus it can be seen from the above experiment that at least lolitrem B has a blocking effect on at least BK channels.

Experiment 2

In this experiment, it is shown that whilst some lolitrem compounds are known to cause tremorgenicity to one extent or another, it is not certain that there is a direct link to BK channel blocking and vice versa.

The experiment uses 31-epilolitrem B, a known non-tremorgenic lolitrem compound as described in Munday-Finch et al 1996.

The same methods were used for testing the antagonist effect of lolitrem B as described in Experiment 1 above.

Preparation of 31-epilolitrem B

The lolitrem derivative 31-epilolitrem B (FIG. 1) was prepared by base-catalysed epimerization of lolitrem B according to S. Munday-Finch, 1997. The lolitrem B was extracted from ryegrass seed infected with Neotyphodium lolii (Gallagher et al., 1981, Miles et al., 1994). A stock of 100 μM 31-epilolitrem B was made up in DMSO. This was diluted to the appropriate concentration in internal solution. The final DMSO concentration was 0.1% for 100 nM compound and did not exceed 0.02% for lower concentrations.

The results showed that 31 -epilolitrem B at a concentration of 100 nM inhibited BK channel currents (α subunit) to 0.06±0.02 (n=7) of the control response. The current response increased to 0.59 of the control response after wash-out with control solution for 26 minutes in one experiment,

Experiment 3

The experiment uses lolitriol, a known non-tremorgenic lolitrem compound as described in Munday-Finch, 1997.

The same methods were used for testing the antagonist effect of lolitrem B as described in Experiment 1 above.

Preparation of Lolitriol

The lolitrem derivative lolitriol was prepared by acid hydrolysis of lolitrem B as reported by Miles et al., 1992. The lolitrem B was extracted from ryegrass seed infected with Neotyphodium lolii (Gallagher et al., 1981, Miles et al., 1994). A stock of 500 μM lolitriol was made up in DMSO. This was diluted to the appropriate concentration in internal solution. The final DMSO concentration was 0.1% for 1 μM lolitriol and did not exceed 0.05% for lower concentrations.

The results showed that lolitriol at a concentration of 100 nM inhibited BK channel currents (α subunit) to 0.29 (n=2) of the control response. 200 nM lolitriol inhibited BK channel currents (α subunit) to 0.25 (n=1) of the control response. The current response increased to 0.86 of the control (n=2) response after wash-out with control solution for 30 minutes at a flow rate of 4 ml/min in three experiments,

The results from Experiments 2 and 3 show that non-tremorgenic lolitrem compounds can inhibit BK channels. Tremorgenicity is thus unlikely to be directly linked to BK channel blocking which is a similar result to that found in general indole diterpene studies (Knaus et al 1994).

Experiment 4

The experiment uses lolitrem E, a known partial-tremorgenic lolitrem compound as described in Munday-Finch, 1997. Lolitrem E is intermediate in structure between lolitrem B and lolitriol.

The same methods were used for testing the antagonist effect of lolitrem E as described in Experiment 1 above.

The results showed that lolitrem E at a concentration of 100 nM inhibited BK channel currents (α subunit) to 0.01 of the control response in one experiment.

Experiment 5

The aim of this experiment was to determine whether the non-tremorgenic lolitrems, 31-epilolitrem B and lolitriol, inhibit function of BK channels that contain an accessory beta subunit. Assuming inhibition was found, it was also an aim to determine if inhibition is effected by the type of beta subunit present.

We used BK channels containing beta subunits that are expressed in smooth muscle (β₃) and brain (β₄). BK channels with subunit combinations α+β₁ or α+β₄ were expressed in human embryonic kidney cells and their function assayed by patch clamping.

Methods

hSlo α subunit large conductance calcium-activated potassium channels in pcDNA3 (Meera et al., 1997) together with the human β₄ subunit in pEGFP-N1 were transiently expressed in human embryonic kidney cells (HEK293). HEK cells were grown in a mix of DMEM (Dulbecco's Modified Eagle Medium) and 2.5 mM HEPES (N-[2-Hydroxyethyl]-piperazine-N′-[2-ethanesulfonic acid]), supplemented with minimal essential amino acids and 10% fetal bovine serum. Cells were subsequently plated onto 24-well plates, grown to 95% confluency and transfected 24 hours later with 10 μg of hSlo, 8 μg of β₄ and 2 μl Lipofectamine 2000™. Cells were plated onto cover slips 24 hours later. Transfected cells expressed green fluorescent protein and were identified under UV light by their fluorescence.

Results

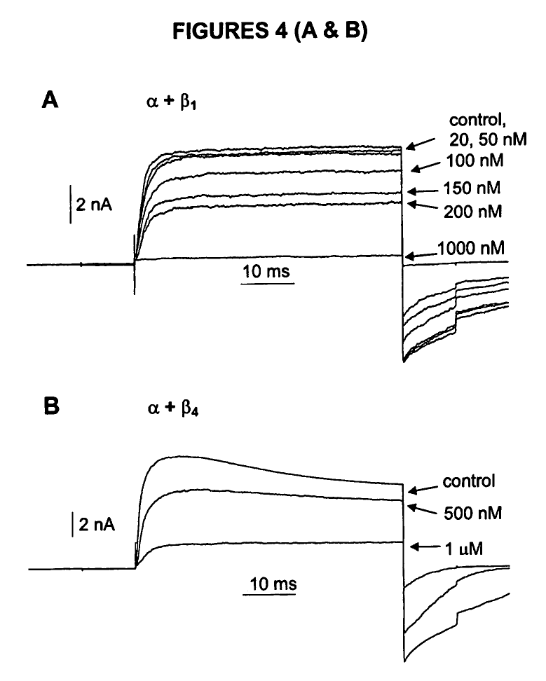

Both α+β₁ and α+β₄ BK channels produced potassium currents in response to depolarising voltages in the presence of 10 μM free calcium (FIG. 4). The maximum current amplitude was typically 1-8 nA. The characteristics of the current responses were similar to that previously reported for these channels (Brenner et al., 2000b, Ahring et al., 1997 Behrens et al., 2001 Lippiat et al., 2003)

31-epilolitrem B and Lolitriol Inhibit BK Channel Function

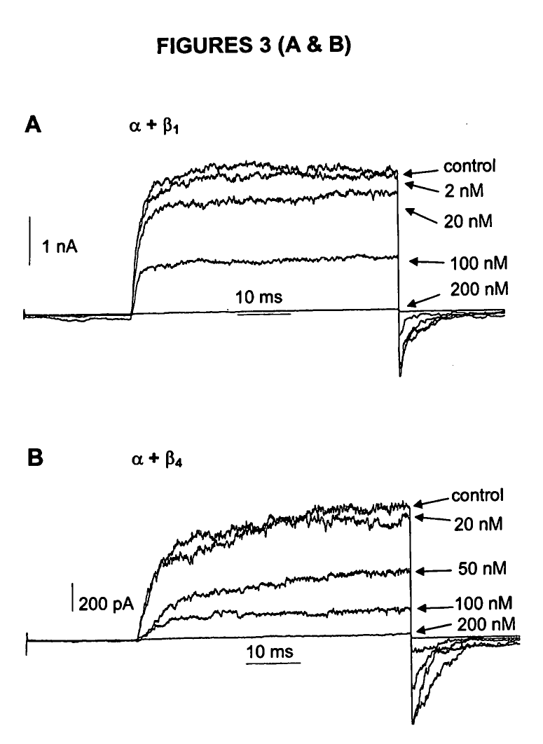

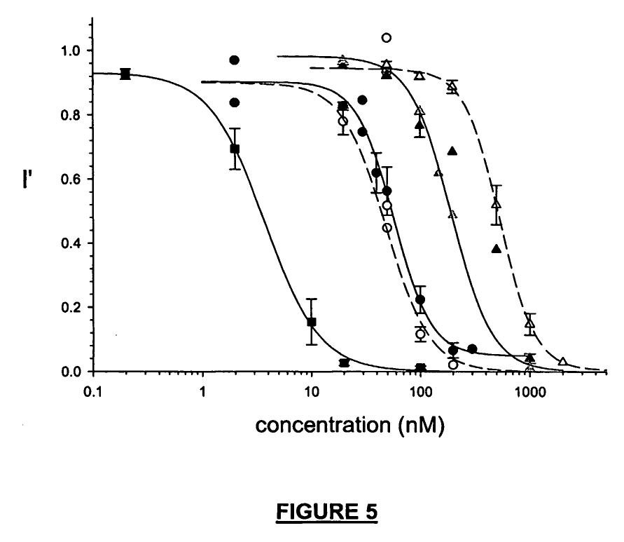

Both 31-epilolitrem B and lolitriol inhibited potassium currents through BK channels but at different concentrations (FIG. 3 and FIG. 4). The differences in the concentration of the lolitrems required to inhibit BK channels is clearly shown in FIG. 5. Dose response data for inhibition of BK channels by lolitrems are summarised below in Table 1. The IC₅₀ for inhibition by 31 -epilolitrem B was lower than that for lolitriol in both α+β₁ (3 times lower) and α+β₄ (10 times lower) BK channels. These results indicated that 31-epilolitrem B has a higher apparent affinity for BK channels than lolitriol in both α+β₁ and α+β₄ BK channels.

TABLE 1
Dose-response data for inhibition of BK channels by lolitrems

| | lolitrem B | | 31-epilolitrem B | | | lolitriol | |
|---|---|---|---|---|---|---|---|
| | IC₅₀ | h | IC₅₀ | | h | IC₅₀ | h |
| α alone | 3.7 ± 0.4 | 1.7 | — | | — | — | — |
| | (n = 11) | | | | | | |
| α + β₁ | — | — | 58 ± 6 | (n = 10) | 2.7 | 195 | 2.3 |
| α + β₄ | — | — | 49 | (n = 5) | 2.2 | 536 ± 16 (n = 7) | 2.7 |

h = Hill coefficient

The above findings show that both tremorgenic and non-tremorgenic lolitrems can inhibit BK channel function. Therefore it is unlikely that BK channels mediate the tremorgenic actions of lolitrems, but rather that other molecular sites are involved.

A study of other indole diterpene compounds by McMillan et al., (2003) compared effects of tremorgenic versus non-tremorgenic compounds on BK channel function and also found differences between the two in their degree of block. The non-tremorgenic paxilline analogue, desoxypaxilline inhibits BK channels but requires 24-times the concentration to produce the same degree of inhibition as for paxilline, which is tremorgenic.

Twice the concentration of lolitriol was required to inhibit α+β₄ channels compared with α+β₁ channels.

Experiment Summary

The above experiments determine the effect of four lolitrems (and by inference other related chemical structures): lolitrem B, lolitrem E, 31-epilolitrem B and lolitriol on the function of BK channels. It is demonstrated that these compounds inhibit potassium currents through BK channels.

The results also show that BK channels that contain accessory beta subunits are also inhibited by lolitrem B, lolitrem E, 31-epilolitrem B and lolitriol.

It is envisaged by the inventors that, because 31-epilolitrem B and lolitriol inhibit BK channel function and are non-tremorgenic in mice, they may have potential as research tools in the study of BK channel pharmacology or as drugs.

The relatively low concentration of 31-epilolitrem B, that is sufficient for BK inhibition, together with its non-tremorgenic properties, suggest uses for this lolitrem derivative in in vivo applications where a BK channel blocker is required.

Aspects of the present invention have been described by way of example only and it should be appreciated that modifications and additions may be made thereto without departing from the scope thereof as defined in the appended claims.

### REFERENCES

Amberg G. C., Bonev A. D., Rossow C. F., Nelson M. T. and Santana L. F. 2003. Modulation of the molecular composition of large conductance, Ca(2+) activated K(+) channels in vascular smooth muscle during hypertension. J Clin Invest. 112, 717-24.
Amberg G. C. and Santana L. F. 2003. Downregulation of the BK channel beta1 subunit in genetic hypertension. Circ Res. 93, 965-71.
Ahring P. K., Strobaek D., Christophersen P., Olesen S. P. and Johansen T. E. 1997. Stable expression of the human large-conductance Ca2+-activated K+channel alpha- and beta-subunits in HEK293 cells. FEBS Lett. 415, 67-70.
Behrens R., Nolting A., Reimann F., Schwarz M., Waldschutz R. and Pongs O. 2000. hKCNMB3 and hKCNMB4, cloning and characterization of two members of the large-conductance calcium-activated potassium channel beta subunit family. FEBS Lett. 474, 99-106.
Brenner R., Perez G. J., Bonev A. D., Eckman D. M., Kosek J. C., Wiler S. W., Patterson A. J., Nelson M. T. and Aldrich R. W. 2000. Vasoregulation by the beta1 subunit of the calcium-activated potassium channel. Nature. 407, 870-6.
Brenner R., Jegla T. J., Wickenden A., Liu Y. and Aldrich R. W. 2000. Cloning and functional characterization of novel large conductance calcium-activated potassium channel beta subunits, hKCNMB3 and hKCNMB4. J Biol Chem. 275, 6453-61.
Gallagher R. T., White E. P. and Mortimer P. H. 1981. Ryegrass staggers: isolation of potent neurotoxins lolitrem A and lolitrem B from staggers-producing pastures. N Z Vet J. 29, 189-90.
Gallagher R. T. and Hawkes A. D. 1986. The potent tremorgenic neurotoxins lolitrem B and aflatrem: a comparison of the tremor response in mice. Experientia. 42, 823-5.
Golding N. L., Jung H. Y., Mickus T. and Spruston N. 1999. Dendritic calcium spike initiation and repolarization are controlled by distinct potassium channel subtypes in CA1 pyramidal neurons. J Neurosci. 19, 8789-98.
Gribkoff V. K., Starrett J. E., Jr. and Dworetzky S. I. 2001a. Maxi-K potassium channels: form, function, and modulation of a class of endogenous regulators of intracellular calcium. Neuroscientist. 7, 166-77.
Gribkoff V. K., Starrett J. E., Jr., Dworetzky S. I., Hewawasam P., Boissard C. G., Cook D. A., Frantz S. W., Heman K., Hibbard J. R., Huston K., Johnson G., Krishnan B. S., Kinney G. G., Lombardo L. A., Meanwell N. A., Molinoff P. B., Myers R. A., Moon S. L., Ortiz A., Pajor L., Pieschl R. L., Post-Munson D. J., Signor L. J., Srinivas N., Taber M. T., Thalody G., Trojnacki J. T., Wiener H. , Yeleswaram K. and Yeola S. W. 2001b. Targeting acute ischemic stroke with a calcium-sensitive opener of maxi-K potassium channels. Nat Med. 7, 471-7.
Gribkoff V. K., Lum-Ragan J. T., Boissard C. G., Post-Munson D. J., Meanwell N. A., Starrett J.E., Jr., Kozlowski E. S., Romine J. L., Trojnacki J. T., McKay M. C., Zhong J. and Dworetzky S. I. 1996. Effects of channel modulators on cloned large-conductance calcium-activated potassium channels. Mol Pharmacol. 50, 206-17.
Hu H., Shao L. R., Chavoshy S., Gu N., Trieb M., Behrens R., Laake P., Pongs O., Knaus H. G., Ottersen O. P. and Storm J. F. 2001. Presynaptic Ca2+-activated K+channels in glutamatergic hippocampal terminals and their role in spike repolarization and regulation of transmitter release. J Neurosci. 21, 9585-97
Kaczorowski G. J. and Garcia M. L. 1999. Pharmacology of voltage-gated and calcium-activated potassium channels. Curr Opin Chem Biol. 3, 448-58.
Kaczorowski G. J., Knaus H. G., Leonard R. J., McManus O. B. and Garcia M. L. 1996. High-conductance calcium-activated potassium channels; structure, pharmacology, and function. J Bioenerg Biomembr. 28, 255-67.
Knaus H. G., McManus O. B., Lee S. H., Schmalhofer W. A., Garcia-Calvo M., Helms L. M., Sanchez M., Giangiacomo K., Reuben J. P., Smith A. B., 3rd and et al. 1994. Tremorgenic indole alkaloids potently inhibit smooth muscle high-conductance calcium-activated potassium channels. Biochemistry. 33, 5819-28.
Lane G., Christensen M. and Miles C. 2000. Coevolution of fungal endophytes with grasses: the significance of secondary metabolites. chapter 14, 341-388.
Lippiat J. D., Standen N. B., Harrow I. D., Phillips S. C. and Davies N. W. 2003. Properties of BK(Ca) channels formed by bicistronic expression of hSloalpha and beta1-4 subunits in HEK293 cells. J Membr Biol 192, 141-8.
Mantle P. G. 1983. Amino acid neurotransmitter release from cerebrocortical synaptosomes of sheep with severe ryegrass staggers in New Zealand. Res Vet Sci. 34, 373-5.
McLeay L. M., Smith B. L. and Munday-Finch S. C. 1999. Tremorgenic mycotoxins paxilline, penitrem and lolitrem B, the non-tremorgenic 31-epilolitrem B and electromyographic activity of the reticulum and rumen of sheep. Res Vet Sci. 66, 119-27.
McMillan L. K., Carr R. L., Young C. A., Astin J. W., Lowe R. G., Parker E. J., Jameson G. B., Finch S. C., Miles C. O., McManus O. B., Schmalhofer W. A., Garcia M. L., Kaczorowski G. J., Goetz M., Tkacz J. S. and Scott B. 2003. Molecular analysis of two cytochrome P450 monooxygenase genes required for paxilline biosynthesis in Penicillium paxilli, and effects of paxilline intermediates on mammalian maxi-K ion channels. Mol Genet Genomics. 270, 9-23.
Meera P., Wallner M., Song M. and Toro L. 1997. Large conductance voltage- and calcium-dependent K+channel, a distinct member of voltage-dependent ion channels with seven N-terminal transmembrane segments (S0-S6), an extracellular N terminus, and an intracellular (S9-S10) C terminus. Proc Natl Acad Sci U S A. 94,14066-71.
Meera P., Wallner M. and Toro L. 2000. A neuronal beta subunit (KCNMB4) makes the large conductance, voltage- and Ca2+-activated K+channel resistant to charybdotoxin and iberiotoxin. Proc Natl Acad Sci U S A. 97, 5562-7.
Miles C. O., Munday-Finch S. C., Wilkins A. L., Ede R. M. and Towers N. R. 1994. Large-scale isolation of lolitrem B and structure determination of lolitrem E. J Agric Food Chem. 42, 1488-1492.
Munday-Finch S. C. 1997. Isolation and structure elucidation of lolilline, a possible biosynthetic precursor of the lolitrem family of tremorgenic mycotoxins. J Agric Food Chem. 45, 199-204.
Munday-Finch S. C. 1997. Aspects of the chemistry and toxicology of indole-diterpenoid mycotoxins involved in tremorgenic disorders of livestock. University of Waikato, New Zealand.
Munday-Finch S. C., Miles C. O., Wilkins A. L. and Hawkes A. D. 1995. Isolation and structure elucidation of lolitrem A, a tremorgenic mycotoxin from perennial ryegrass infected with Acremonium lolii. J Agric Food Chem. 43, 1283-1288.
Munday-Finch S. C., Wilkins A. L., Miles C. O., Ede R. M. and Thomson R. A. 1996. Structure elucidation of lolitrem F, a naturally occurring sterioisomer of the tremorgenic mycotoxin lolitrem B, isolated from Lolium perenne infected with Acremonium lolii. J Agric Food Chem. 44, 2782-2788.
Munday-Finch S. C., Wilkins A. L. and Miles C. O. 1998. Isolation of Lolicine A, Lolicine B, Lolitriol, and Lolitrem N from Lolium perenne Infected with Neotyphodium lolii and Evidence for the Natural Occurrence of 31-Epilolitrem N and 31-Epilolitrem F. J Agric Food Chem. 46, 590-598.
Nelson M. T., Cheng H., Rubart M., Santana L. F., Bonev A. D., Knot H. J. and Lederer W. J. 1995. Relaxation of arterial smooth muscle by calcium sparks. Science. 270, 633-7.
Orio P., Rojas P., Ferreira G. and Latorre R. 2002. New disguises for an old channel: MaxiK channel beta-subunits. News Physiol Sci. 17, 156-61.
McLeay L. M. and Smith B. L. 1999. Effects of the mycotoxins lolitrem B and paxilline on gastrointestinal smooth muscle, the cardiovascular and respiratory systems, and temperature in sheep. Grasslands Research and Practice Series. 7, 69-75.
Sanchez M. and McManus O. B. 1996. Paxilline inhibition of the alpha-subunit of the high-conductance calcium-activated potassium channel. Neuropharmacology. 35, 963-8.
Shao L. R., Halvorsrud R., Borg-Graham L. and Storm J. F. 1999. The role of BK-type Ca2+-dependent K+channels in spike broadening during repetitive firing in rat hippocampal pyramidal cells. J Physiol. 521 Pt 1,135-46.
Smith B. L., McLeay L. M. and Embling P. P. 1997. Effect of the mycotoxins penitrem, paxilline and lolitrem B on the electromyographic activity of skeletal and gastrointestinal smooth muscle of sheep. Res Vet Sci. 62, 111-6.
Wang L., Cross A. L., Allen K. L. Smith B. L. and McLeay L. M. 2003. Tremorgenic mycotoxins increase gastric smooth muscle activity of sheep reticulum and rumen in vitro. Res Vet Sci. 74, 93-100.
NZ 236879 (1991) Miles et al, AgResearch Limited, Lolitrems—antibodies and assay techniques.
U.S. Pat. No. 4,973,601 (1990) Dowd et al, Control of insects by fungal tremorgenic mycotoxins.
U.S. Pat. No. 5,541,208 (1996) Garcia et al, Indole Diterpene Alkaloid Compounds.

1. A method of preventing repolarisation or hyperpolarisation of a cell, wherein the cell contains a BK channel, including the administration to the cell of at least one pharmacologically effective amount of composition containing a BK channel antagonist containing the moiety shown in structure (I): 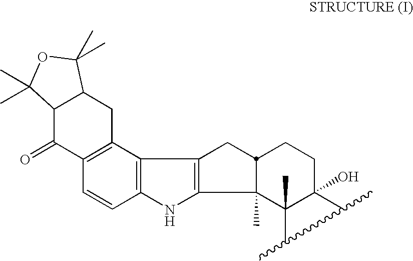
or derivatives thereof.

2. The method as claimed in claim 1 wherein the derivatives of structure (I) are selected from the group consisting of: salts, analogues, isomers, and combinations thereof.

3. The method as claimed in claim 1 wherein the antagonist compound is selected from the group consisting of: lolitrem B, lolitrem A, lolitrem F, 31-epilolitrem F, 31-epilolitrem B, lolitrem E, lolitrem E acetate, lolitrem L, lolitrem G, lolitrem C, lolitrem M, lolitriol, lolitriol acetate, lolitrem N, lolitrem J, lolitrem H, lolitrem K, lolicine A and B, 30-desoxy lolitrem B-30α-ol, 30-desoxy-31-epilolitrem B-30α-ol, 30-desoxylolitrem B-30-ene lolilline and combinations thereof.

4. The method as claimed in claim 1 wherein the antagonist compound is selected from the group consisting of: 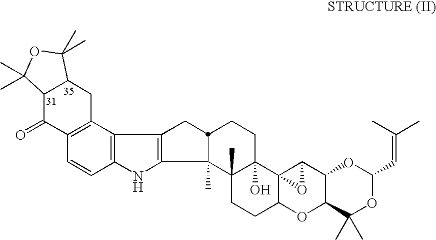
which includes compounds selected from the group consisting of: lolitrem B=31α, 35β stereochemistry; 31-epilolitrem B=31β, 35β stereochemistry; lolitrem F=31α, 35α; 31-epilolitrem F=31β, 35α; 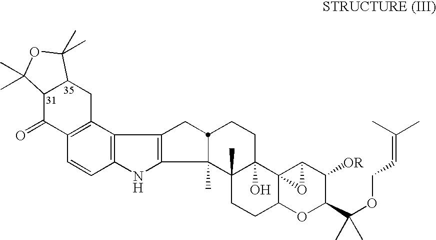
which includes compounds selected from the group consisting of: lolitrem E=31α, 35β stereochemistry where R=H or acetate; lolitrem L=31α, 35α stereochemistry where R=H or acetate; 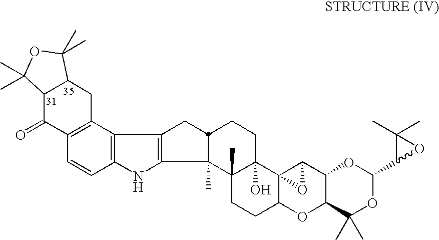
which includes compounds selected from the group consisting of: lolitrem A=31α, 35β stereochemistry; lolitrem G=31α, 35α stereochemistry; 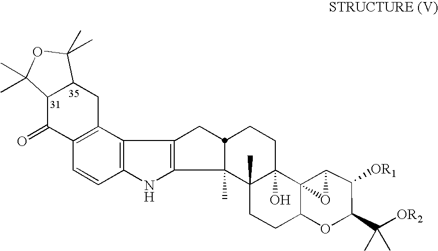
which includes compounds selected from the group consisting of: lolitriol;=31α, 35β stereochemistry where R₁=H or acetate and R₂=H; lolitrem N=31α, 35α stereochemistry where R₁=H or acetate and R₂=H; Lolitrem J=31α, 35β stereochemistry where R₁=H or acetate and R₂=acetate; 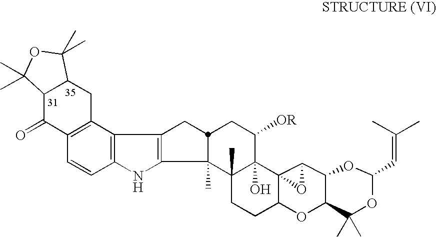
which includes lolitrem H=31α, 35β stereochemistry where R=H or acetate; 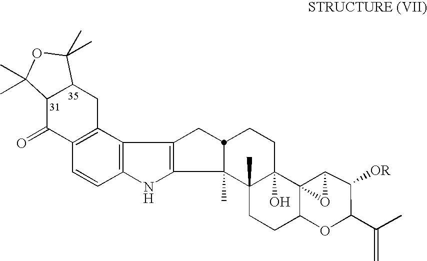
which includes lolitrem K=31α, 35β stereochemistry, where R=H or acetate; 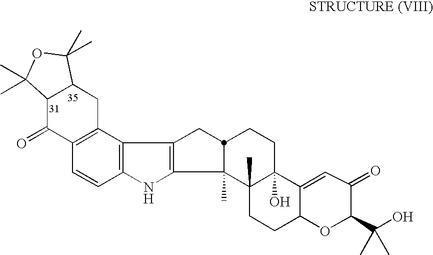
which includes lolilline=31α, 35β stereochemistry; 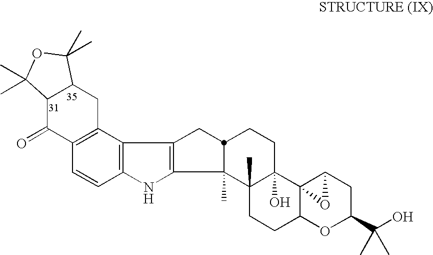
which includes lolitrem M=31α, 35β stereochemistry; 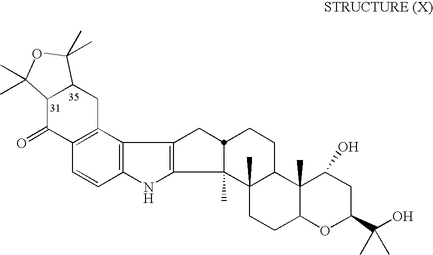
which includes lolicine A=31α, 35β stereochemistry; 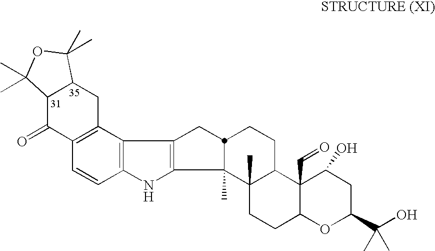
which includes lolicine B=31α, 35β stereochemistry; 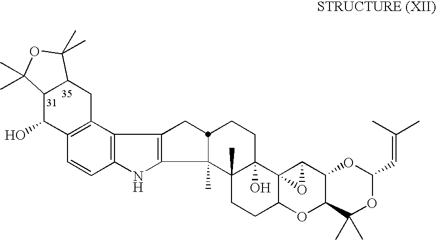
which includes compounds selected from the group consisting of: 30-desoxylolitrem B-30α-ol=31α, 35β stereochemistry; 30-desoxy-31-epilolitrem B-30α-ol =31α, 35β stereochemistry; 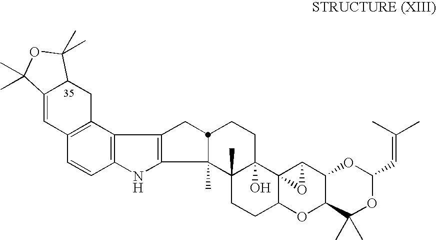
which includes 30-desoxylolitrem B-30-ene=35β stereochemistry; and
combinations of the above compounds.

5. The method as claimed in claim 1 wherein the composition further includes pharmaceutically and physiologically acceptable carriers.

6. The method as claimed in claim 5, wherein the pharmaceutically and physiologically acceptable carriers include components selected from the group including; fillers; excipients; modifiers; humectants; stabilisers; emulsifiers; diluents; and other formulation components such as a use of a lipid vehicle.

7. The method as claimed in claim 1, wherein the composition is administered in a form selected from the group including: an injection; a tablet; a capsule; a suppository; an injection; a suspension; a drink or tonic; a syrup; a powder; an ingredient in solid or liquid foods; a nasal spray; a sublingual wafer; a transdermal patch; a transdermal injection; and combinations thereof.

8. The method as claimed in claim 1, wherein the BK channel antagonist compound or compounds are extracted from endophyte-infected plants and seeds.

9. The method as claimed in claim 1, wherein the BK channel antagonist compound or compounds are extracted from fungal cultures.

10. The method as claimed in claim 1, wherein the BK channel antagonist compound or compounds are derived by chemical synthesis.

11. The method as claimed in claim 1, wherein the BK channel antagonist compound or compounds are extracted from heterologous expression systems.

12. The method as claimed in claim 8 wherein the perennial ryegrass seed is from Lolium perenne.

13. The method as claimed in claim 1, wherein the BK channel antagonist compound or compounds has activity against both alpha (α) subunit and alpha plus beta (β) accessory subunit (β₁ to β₄) channels.

14. The method as claimed in claim 1, wherein, for lolitrem B, the degree of antagonist inhibition is approximately 97% for a composition containing approximately 20 nM lolitrem B.

15. The method as claimed in claim 1, wherein, for lolitrem B, the half maximal degree of antagonist inhibition (IC₅₀) is found for a composition containing approximately 3.7±0.4 nM of lolitrem B.

16. The method as claimed in claim 1, wherein, for lolitriol, the degree of antagonist inhibition is approximately 100% for a composition containing approximately 1000 nM lolitriol.

17. The method as claimed in claim 1, wherein, for lolitriol, the half maximal degree of antagonist inhibition (IC₅₀) is found for a composition containing approximately 195 nM of lolitriol to inhibit α and β₁ BK channel activity

18. The method as claimed in claim 1, wherein, for lolitriol, the half maximal degree of antagonist inhibition (IC₅₀) is found for a composition containing approximately 536±16 nM of lolitriol to inhibit α and β₄ activity.

19. The method as claimed in claim 1, wherein, for 31-epilolitrem B, the degree of antagonist inhibition is approximately 100% for a composition containing approximately 200 nM 31-epilolitrem B.

20. The method as claimed in claim 1, wherein, for 31-epilolitrem B, the half maximal degree of antagonist inhibition (IC₅₀) is found for a composition containing approximately 58±6 nM of 31-epilolitrem B to inhibit α and β₁ activity.

21. The method as claimed in claim 1 wherein, for 31-epilolitrem B, the half maximal degree of antagonist inhibition (IC₅₀) is found for a composition containing approximately 49 nM of 31-epilolitrem B to inhibit α and β₄ activity.

22. The method as claimed in claim 1, wherein, for lolitrem E, the degree of antagonist inhibition is approximately 100% for a composition containing approximately 100 nM lolitrem E.

23. The method as claimed in claim 1, wherein the antagonist effect of the composition is not able to be reversed by wash out for concentrations of 10 nM or greater of lolitrem B compound.

24-46. (canceled)

47. A composition that contains a pharmacologically effective amount of at least one BK channel antagonist compound containing the moiety shown in structure (VII): 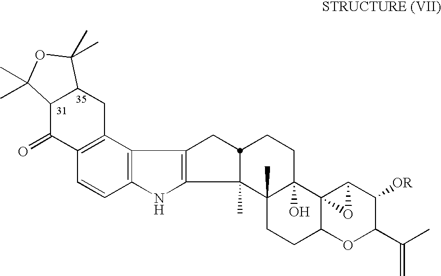
which includes lolitrem K=31α, 35β stereochemistry, where R=H or acetate.

48. A composition that contains a pharmacologically effective amount of at least one BK channel antagonist compound containing the moiety shown in structure (IX): 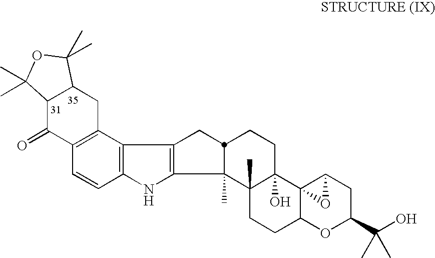
which includes lolitrem M=31α, 35β stereochemistry.

49. A composition that contains a pharmacologically effective amount of at least one BK channel antagonist compound containing the moiety shown in structure (XII): 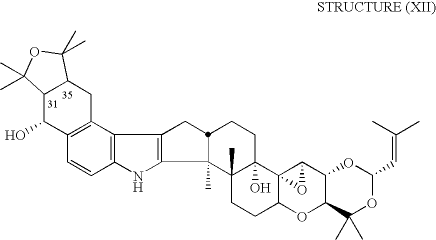
which includes compounds selected from the group consisting of: 30-desoxylolitrem B-30α-ol=31α, 35β stereochemistry; 30-desoxy-31-epilolitrem B-30α-ol=31β, 35β stereochemistry.

50. A composition that contains a pharmacologically effective amount of at least one BK channel antagonist compound wherein the antagonist compound is structure (XIII): 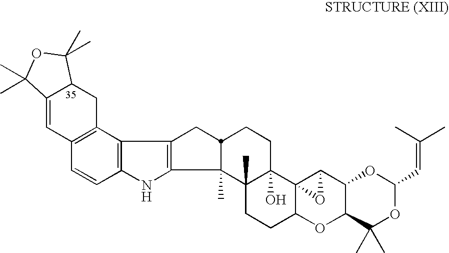
which includes 30-desoxylolitrem B-30-ene=35β stereochemistry.

51. The method as claimed in claim 11 wherein the heterologous expression system is selected from the group consisting of bacteria, yeast, fungi, plants, and animal cells.
